(19) United States
(12) Patent Application Publication
(10) Pub. No.: US 2016/0219801 A1
(43) Pub. Date: Aug. 4, 2016

(54) METHOD OF REGENERATING RUBBER TREE, METHOD OF PROPAGATING RUBBER TREE, METHOD OF INDUCING SHOOT, METHOD OF ELONGATING SHOOT, METHOD OF ROOTING SHOOT, AND METHOD OF ACCLIMATIZING YOUNG PLANT

(71) Applicant: SUMITOMO RUBBER INDUSTRIES, LTD., Kobe-shi (JP)

(72) Inventors: Akari Okada, Kobe-shi (JP); Sompong Techato, Hat Yai (TH)

(73) Assignee: SUMITOMO RUBBER INDUSTRIES, LTD., Kobe-shi (JP)

(21) Appl. No.: 15/000,843

(22) Filed: Jan. 19, 2016

(30) Foreign Application Priority Data
Feb. 3, 2015 (JP) 2015-019436

(51) Int. Cl. A01G 23/04; A01G 1/00; C12N 5/04

(52) U.S. Cl. CPC A01G 23/04; C12N 5/04; A01G 1/001

(57) ABSTRACT

Provided are a method of regenerating a rubber tree that can stably regenerate the rubber tree from a tissue including a node, axillary bud or apical bud derived from a mature rubber tree, a young rubber tree, or a plantlet of a rubber tree; and a method of propagating a rubber tree that can stably acquire large numbers of clone seedlings of the rubber tree. Included is a method of regenerating a rubber tree including an induction step of culturing a tissue including a node, axillary bud or apical bud of a rubber tree in an induction medium containing a plant growth hormone and a carbon source to form a shoot.

### TECHNICAL FIELD

The present invention relates to a method of regenerating a rubber tree, a method of propagating a rubber tree, a method of inducing a shoot, a method of elongating a shoot, a method of rooting a shoot, and a method of acclimatizing a young plant.

### BACKGROUND ART

At present, natural rubber (a type of polyisoprenoid) used in industrial rubber products is obtained by cultivating rubber-producing plants such as Para rubber tree (Hevea brasiliensis) of the family Euphorbiaceae or Indian rubber tree (Ficus elastica) of the family Moraceae, biosynthesizing natural rubber in laticifer cells of the plants, and manually collecting the natural rubber from the plants.

At present, Hevea brasiliensis is practically the only one source of natural rubber for industrial use. It is also used as a main material of rubber products for various applications widely and in large quantities. However, Hevea brasiliensis is a plant that can grow only in limited areas such as in Southeast Asia and South America. Moreover, Hevea brasiliensis requires about seven years from planting to mature enough for rubber extraction, and the extraction season may be limited. Furthermore, the period during which natural rubber can be extracted from mature trees is limited to 20 to 30 years.

As the demand for natural rubber is expected to rise in the future mainly in developing countries, there are concerns about the depletion of natural rubber resources. Thus, stable natural rubber sources are desired.

In this context, there have been attempts to increase production of natural rubber using Hevea brasiliensis. Plantlets of Hevea brasiliensis are propagated by growing seedlings after sowing, and preparing stocks from the grown seedlings, and then grafting buds obtained from clone seedlings onto the stocks. Since the buds that can be obtained from clone seedlings are limited, it is necessary to mass-propagate superior clone seedlings to spread superior varieties.

In addition, there is a possibility that the grafted buds obtained from clone seedlings according to the conventional clonal propagation techniques may simultaneously inherit diseases of the original trees, and diseased seedlings may be propagated. However, there is no possibility of acquiring diseases during grafting if the clone seedlings are aseptically propagated by mass-propagating the seedlings through tissue culture.

Large quantities of grafts are used to propagate clone seedlings of Hevea brasiliensis using the method of grafting from clone seedlings. It is considered that the number of buds that can be used from one clone seedling is limited. For this reason, it is necessary to stably obtain grafts for mass propagation of clone seedlings.

Moreover, grafts are not true clone seedlings as they may be affected by stocks. In this regard, it is possible to produce true clone seedlings by propagating shoots and rooting the shoots.

Meanwhile, in order to increase the yield of isoprenoids in plants, for example, methods of modifying plants so as to improve stress resistance or to increase the amount of isoprenoids accumulated in the plants can be considered. Although methods using artificial crossing or mutations can be considered as the methods for modifying plants, they have difficulty in efficiently imparting desired properties and thus have low feasibility. For this reason, for the modification of plants, it is considered to use a cellular engineering approach in which a target gene is introduced into plant cells to impart desired properties.

In the case of the cellular engineering approach, it is necessary to allow plant cells or tissue into which a target gene is introduced to re-differentiate or regenerate into plants. In the related art, studies on tissue culture in various plants have been made and a method of inducing shoots from plant calli has been known (the culture conditions for regenerating plants from calli vary depending on the type of plant). However, there have been few studies on the tissue culture conditions for stably regenerating and propagating various isoprenoid-producing plants and it has been difficult to stably regenerate and propagate isoprenoid-producing plants, including rubber trees, by tissue culture.

### SUMMARY OF INVENTION

### Technical Problem

In general, tissue culture is carried out by changing the type or concentration of used plant growth regulating substance in each of the stages of shoot formation, elongation, and rooting. However, in the conventional culture methods, it is necessary to combine many plant growth regulating substances for use in the medium, and these methods leave something to be desired as culture methods.

In the meantime, in the conventional methods of propagating Hevea brasiliensis, clone seedlings are propagated by grafting buds obtained from clone seedlings onto stocks, but the buds that can be used from clone seedlings are limited. For this reason, it is difficult to rapidly mass-propagate clone seedlings and there is a need to develop techniques for propagating superior clone seedlings.

The present invention aims to solve the problems and provide a method of regenerating a rubber tree that can stably regenerate the rubber tree from a tissue including a node, axillary bud or apical bud derived from a mature rubber tree, a young rubber tree, or a plantlet (e.g., a graft, seedling, layer, or cutting) of a rubber tree; and a method of propagating a rubber tree that can stably acquire large numbers of clone seedlings of the rubber tree. The present invention also aims to provide a method of stably inducing a shoot of a rubber tree, a method of stably elongating a shoot of a rubber tree, a method of stably rooting a shoot of a rubber tree, and a method of stably acclimatizing a young rubber tree.

### Solution to Problem

The present inventors have conducted extensive studies and, as a result, they have found that by culturing a tissue including a node, axillary bud or apical bud derived from a mature rubber tree, a young rubber tree, or a plantlet of a rubber tree in an induction medium containing a plant growth hormone and a carbon source, a shoot can be stably formed and then can be stably elongated, rooted, and acclimatized. In other words, the present invention is accomplished based on the findings that by culturing a tissue including a node, axillary bud or apical bud of a rubber tree in an induction medium containing a plant growth hormone and a carbon source to form a shoot, it is possible to stably regenerate the rubber tree and obtain clone seedlings of the rubber tree, and further to stably propagate the clone seedlings of the rubber tree to acquire them in large numbers.

Specifically, the present invention relates to a method of regenerating a rubber tree, including an induction step of culturing a tissue including a node, axillary bud or apical bud of a rubber tree in an induction medium containing a plant growth hormone and a carbon source to form a shoot.

The regeneration method preferably further includes an elongation step of culturing the shoot formed through the induction step in an elongation medium containing a plant growth hormone and a carbon source to elongate the shoot.

The regeneration method preferably further includes a rooting step of culturing the shoot formed through the induction step or the shoot elongated through the elongation step in a root induction medium containing a plant growth hormone and a carbon source to root the shoot.

The regeneration method preferably further includes an acclimatization step of treating the shoot rooted through the rooting step with a disinfectant, followed by transplanting the treated shoot into a cultivation soil and acclimatizing the transplanted shoot.

In the regeneration method, it is preferable that the rubber tree be a plant belonging to the genus Hevea.

In the regeneration method, it is preferable that the induction medium contain 0.01 mass % to 0.1 mass % of activated carbon.

In the regeneration method, it is preferable that the induction medium contain 0.1 mg/L to 5.0 mg/L of silver nitrate.

In the regeneration method, it is preferable that the root induction medium contain 0.01 mass % to 0.1 mass % of activated carbon.

In the regeneration method, it is preferable that the root induction medium contain 0.1 mg/L to 5.0 mg/L of silver nitrate.

In the regeneration method, it is preferable that the disinfectant be an iodine-based disinfectant.

In the regeneration method, it is preferable that the acclimatization step include, after transplanting the rooted shoot treated with the disinfectant into the cultivation soil, gradually exposing the transplanted shoot to a natural environment to adapt the shoot to the environment for acclimatization.

The present invention also relates to a method of propagating a rubber tree, including an induction step of culturing a tissue including a node, axillary bud or apical bud of a rubber tree in an induction medium containing a plant growth hormone and a carbon source to form a shoot.

The propagation method preferably further includes an elongation step of culturing the shoot formed through the induction step in an elongation medium containing a plant growth hormone and a carbon source to elongate the shoot.

The propagation method preferably further includes a propagation step of collecting the shoot formed through the induction step or the shoot elongated through the elongation step, dividing the shoot into multiple shoots, and then culturing the divided shoot in an induction medium containing a plant growth hormone and a carbon source to form a shoot.

The propagation method preferably further includes a rooting step of culturing the shoot formed through the propagation step in a root induction medium containing a plant growth hormone and a carbon source to root the shoot.

The propagation method preferably further includes an acclimatization step of treating the shoot rooted through the rooting step with a disinfectant, followed by transplanting the treated shoot into a cultivation soil and acclimatizing the transplanted shoot.

In the propagation method, it is preferable that the rubber tree be a plant belonging to the genus Hevea.

In the propagation method, it is preferable that the induction medium contain 0.01 mass % to 0.1 mass % of activated carbon.

In the propagation method, it is preferable that the induction medium contain 0.1 mg/L to 5.0 mg/L of silver nitrate.

In the propagation method, it is preferable that the root induction medium contain 0.01 mass % to 0.1 mass % of activated carbon.

In the propagation method, it is preferable that the root induction medium contain 0.1 mg/L to 5.0 mg/L of silver nitrate.

In the propagation method, it is preferable that the disinfectant be an iodine-based disinfectant.

In the propagation method, it is preferable that the acclimatization step include, after transplanting the rooted shoot treated with the disinfectant into the cultivation soil, gradually exposing the transplanted shoot to a natural environment to adapt the shoot to the environment for acclimatization.

The present invention also relates to a method of inducing a shoot of a rubber tree, including culturing a tissue including a node, axillary bud or apical bud of a rubber tree in an induction medium containing a plant growth hormone and a carbon source to form a shoot.

The present invention also relates to a method of elongating a shoot of a rubber tree, including culturing a shoot of a rubber tree in an elongation medium containing a plant growth hormone and a carbon source to elongate the shoot.

The present invention also relates to a method of rooting a shoot of a rubber tree, including culturing a shoot of a rubber tree in a root induction medium containing a plant growth hormone and a carbon source to root the shoot.

The present invention also relates to a method of acclimatizing a young rubber tree, including treating a young rubber tree with a disinfectant, followed by transplanting the treated young rubber tree into a cultivation soil and acclimatizing the transplanted young rubber tree.

### Advantageous Effects of Invention

Since the method of regenerating a rubber tree of the present invention includes an induction step of culturing a tissue including a node, axillary bud or apical bud of a rubber tree in an induction medium containing a plant growth hormone and a carbon source to form a shoot, it is possible to stably regenerate the rubber tree and obtain clone seedlings of the rubber tree. Moreover, since the method of propagating a rubber tree of the present invention includes an induction step of culturing a tissue including a node, axillary bud or apical bud of a rubber tree in an induction medium containing a plant growth hormone and a carbon source to form a shoot, it is possible to stably propagate clone seedlings of the rubber tree and acquire them in large numbers. Furthermore, the method of inducing a shoot of a rubber tree of the present invention can stably induce the shoot of the rubber tree, the method of elongating a shoot of a rubber tree of the present invention can stably elongate the shoot of the rubber tree, the method of rooting a shoot of a rubber tree of the present invention can stably root the shoot of the rubber tree, and the method of acclimatizing a young rubber tree of the present invention can stably acclimatize the young rubber tree. Accordingly, these methods can contribute to mass propagation and molecular breeding of rubber trees.

### BRIEF DESCRIPTION OF DRAWINGS

FIGS. 1(a) to 1(d) are photographs respectively showing the states of (a) a shoot formed through an induction step; (b) a axillary bud left in the division of the shoot in a propagation step; (c) multiple shoots formed through the induction step; and (d) a shoot formed from the divided shoot through the propagation step, of Hevea brasiliensis.

FIGS. 2(a) to 2(c) are photographs respectively showing the states of (a) shoots before rooting in a rooting step; (b) shoots after rooting; and (c) a complete plant obtained after rooting, of Hevea brasiliensis.

FIGS. 3(a) to 3(c) are photographs respectively showing the states of (a) young plants before acclimatization in an acclimatization step; (b) young plants during acclimatization; and (c) young plants one week after the start of acclimatization, of Hevea brasiliensis.

FIG. 4 is a flowchart illustrating the flow of a series of steps of examples of methods of regenerating or propagating a rubber tree of the present invention.

### DESCRIPTION OF EMBODIMENTS

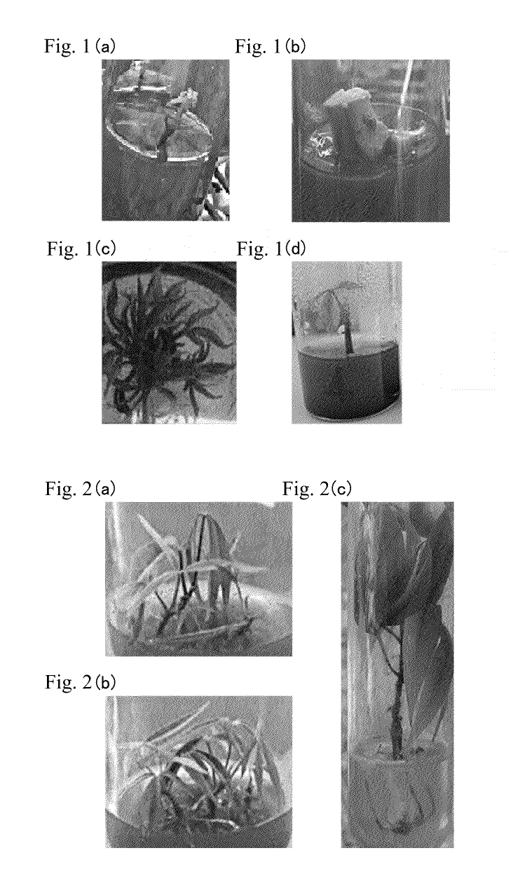

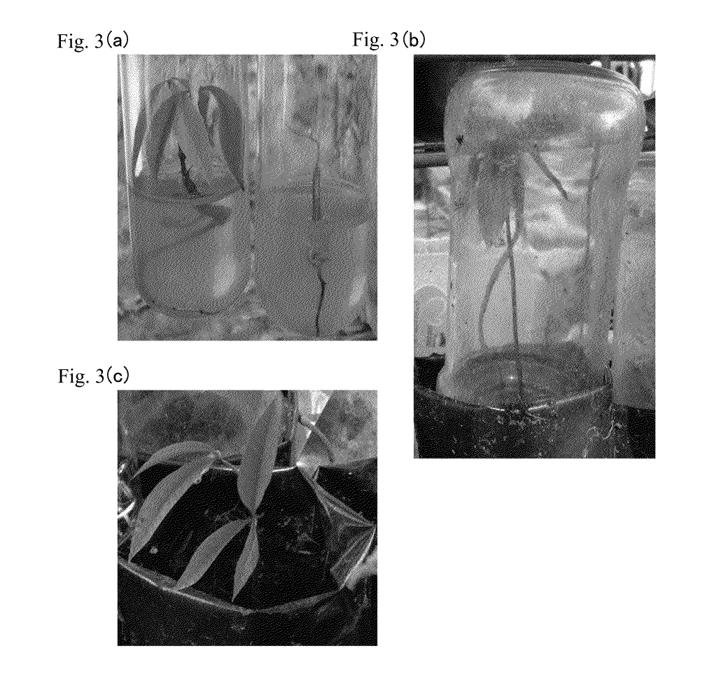

The method of regenerating a rubber tree of the present invention includes an induction step of culturing a tissue including a node, axillary bud or apical bud of a rubber tree in an induction medium containing a plant growth hormone and a carbon source to form a shoot. As described above, by inducing a shoot from a tissue including a node, axillary bud or apical bud of a rubber tree using a specific induction medium, the shoot can be stably formed (see FIG. 1(a)) and thus a tissue (shoot or multiple shoot) that can be used as a graft can be obtained (see FIGS. 1(a) and 1(c)). Further, by elongating, rooting (see FIG. 2), and acclimatizing (see FIG. 3) the formed shoot, it is possible to stably regenerate the rubber tree to obtain clone seedlings of the rubber tree.

Moreover, the method of propagating a rubber tree of the present invention includes an induction step of culturing a tissue including a node, axillary bud or apical bud of a rubber tree in an induction medium containing a plant growth hormone and a carbon source to form a shoot. Since the method of propagating a rubber tree of the present invention is based on the method of regenerating a rubber tree of the present invention described above, it is possible to stably propagate clone seedlings of the rubber tree. More specifically, the shoot formed through the induction step is collected without collecting the node, axillary bud and apical bud portions, and divided into multiple shoots (see FIG. 1(b)), and the obtained tissue can be further cultured in an induction medium to form a shoot (see FIG. 1(d)). Thus, it is possible to increase the number of shoots to mass-propagate a tissue that can be used as a graft, thereby stably acquiring large numbers of clone seedlings of the rubber tree.

The methods of the present invention can be applied to any plant as long as it is a rubber tree from which rubber can be extracted as a resource, and examples include the genus Hevea such as Para rubber tree (Hevea brasiliensis); the genus Sonchus such as Sonchus oleraceus, Sonchus asper, Sonchus brachyotus and Sonchus arvensis; the genus Solidago such as Solidago altissima, Solidago virgaurea subsp. asiatica, Solidago virgaurea subsp. leipcarpa, Solidago virgaurea subsp. leipcarpa f. paludosa, Solidago virgaurea subsp. gigantea, and Solidago gigantea Ait. var. leiophylla Fernald; the genus Helianthus such as sunflower (Helianthus annuus), Helianthus argophyllus, Helianthus atrorubens, Helianthus debilis, Helianthus decapetalus, and Helianthus giganteus; the genus Taraxacum such as dandelion (Taraxacum), Taraxacum venustum H. Koidz, Taraxacum hondoense Nakai, Taraxacum platycarpum Dahlst., Taraxacum japonicum, Taraxacum officinale Weber, and Russian dandelion (Taraxacum koksaghyz); the genus Ficus such as fig (Ficus carica), Indian rubber tree (Ficus elastica), Ficus pumila L., Ficus erecta Thumb., Ficus ampelas Burm. f., Ficus benguetensis Merr., Ficus irisana Elm., Ficus microcarpa L. f., Ficus septica Burm. f., and Indian banyan (Ficus benghalensis); Guayule (Parhenium argentatum); and lettuce (Lactuca serriola). Preferred among these are plants belonging to the family Asteraceae such as plants of the genera Sonchus, Solidago, Helianthus, and Taraxacum; and plants belonging to the family Euphorbiaceae such as plants of the genus Hevea. More preferred are plants belonging to the genus Hevea, particularly preferably Para rubber tree (Hevea brasiliensis).

Hereinafter, the method of propagating a rubber tree of the present invention will be described in detail. Since the method of propagating a rubber tree of the present invention is basically based on the method of regenerating a rubber tree of the present invention, except for including a propagation step which will be described later, it is considered that the description of the method of propagating a rubber tree of the present invention also provides description of the method of regenerating a rubber tree of the present invention. Moreover, since the method of inducing a shoot of a rubber tree of the present invention corresponds to an induction step which will be described later; the method of elongating a shoot of a rubber tree of the present invention corresponds to an elongation step which will be described later; the method of rooting a shoot of a rubber tree of the present invention corresponds to a rooting step which will be described later; and the method of acclimatizing a young rubber tree of the present invention corresponds to an acclimatization step which will be described later, it is considered that the description of the method of propagating a rubber tree of the present invention also provides description of these methods.

The method of propagating a rubber tree of the present invention includes an induction step of culturing a tissue including a node, axillary bud or apical bud of a rubber tree in an induction medium containing a plant growth hormone and a carbon source to form a shoot. Specifically, it may include inducing a shoot from a tissue including a node, axillary bud or apical bud of a rubber tree and propagating the shoot, thereby propagating clone seedlings of the rubber tree. More specifically, it may include inducing a shoot from a tissue including a node, axillary bud or apical bud of a rubber tree, and dividing the shoot into multiple shoots, followed by culturing the divided shoot to form a shoot, and rooting and acclimatizing the formed shoot to regenerate the rubber tree, thereby propagating clone seedlings of the rubber tree.

Specifically, the method of propagating a rubber tree of the present invention preferably includes: an induction step of culturing a tissue including a node, axillary bud or apical bud of a rubber tree in an induction medium containing a plant growth hormone and a carbon source to form a shoot; a propagation step of collecting the shoot formed through the induction step, dividing the shoot into multiple shoots, and then culturing the divided shoot in an induction medium containing a plant growth hormone and a carbon source to form a shoot; a rooting step of culturing the shoot formed through the propagation step in a root induction medium containing a plant growth hormone and a carbon source to root the shoot; and an acclimatization step of treating the shoot rooted through the rooting step with a disinfectant, followed by transplanting the treated shoot into a cultivation soil and acclimatizing the transplanted shoot. More preferably, the method of propagating a rubber tree of the present invention includes: an induction step of culturing a tissue including a node, axillary bud or apical bud of a rubber tree in an induction medium containing a plant growth hormone and a carbon source to form a shoot; an elongation step of culturing the shoot formed through the induction step in an elongation medium containing a plant growth hormone and a carbon source to elongate the shoot; a propagation step of collecting the shoot elongated through the elongation step, dividing the shoot into multiple shoots, and then culturing the divided shoot in an induction medium containing a plant growth hormone and a carbon source to form a shoot; a rooting step of culturing the shoot formed through the propagation step in a root induction medium containing a plant growth hormone and a carbon source to root the shoot; and an acclimatization step of treating the shoot rooted through the rooting step with a disinfectant, followed by transplanting the treated shoot into a cultivation soil and acclimatizing the transplanted shoot. Further, after the propagation step, an elongation step of culturing the formed shoot in an elongation medium containing a plant growth hormone and a carbon source to elongate the shoot may be carried out before the rooting step. That is, the method of propagating a rubber tree of the present invention preferably includes the induction step, the propagation step, the rooting step, and the acclimatization step, and more preferably includes the induction step, the elongation step, the propagation step, the rooting step, and the acclimatization step. Further, the elongation step may be carried out after the propagation step but before the rooting step.

Moreover, the method of regenerating a rubber tree of the present invention preferably includes the induction step, a rooting step of culturing the shoot formed through the induction step in a root induction medium containing a plant growth hormone and a carbon source to root the shoot, and the acclimatization step. More preferably, the method of regenerating a rubber tree of the present invention includes the induction step, the elongation step, a rooting step of culturing the shoot elongated through the elongation step in a root induction medium containing a plant growth hormone and a carbon source to root the shoot, and the acclimatization step.

Each step may be carried out once or plural times by subculture.

Hereinafter, the steps will be described. Although a case where Hevea brasiliensis is used as an example of rubber tree is described in the following, rubber trees other than Hevea brasiliensis can be used to perform the steps in the same manner.

### (Induction Step)

The induction step includes culturing a tissue including a node, axillary bud or apical bud of a rubber tree in an induction medium containing a plant growth hormone and a carbon source to induce and form a shoot.

As the material for inducing the shoot, a tissue including a node, axillary bud or apical bud of a rubber tree is used, and specific examples include tissues including a node, axillary bud or apical bud derived from a mature or young tree, a plantlet, a clone seedling, or a seedling grown aseptically in vitro (aseptic seedling). In the case of the tissues including a node, axillary bud or apical bud derived from a mature or young tree, a plantlet, or a clone seedling, they can be used after they are cut to required sizes as appropriate and then the surface is disinfected or sterilized. However, in the case of the tissues including a node, axillary bud or apical bud derived from a seedling grown aseptically in vitro (aseptic seedling), they can be used after cut to required sizes as appropriate.

In the case of the tissues including a node, axillary bud or apical bud derived from a mature or young tree, a plantlet, or a clone seedling, the surface of the tissues is first washed before culturing in the induction medium. For example, the surface may be washed using a cleanser or a soft sponge, but is preferably washed with running water. The water for washing may contain about 0.1 mass % of a surfactant.

Next, the tissues are disinfected or sterilized. The disinfection or sterilization may be carried out using well-known disinfectants or sterilizers. Preferred are ethanol, benzalkonium chloride, and aqueous sodium hypochlorite. The tissues may be further washed with sterilized water after the disinfection or sterilization treatment.

For example, the following procedures may be used as specific examples of the washing, disinfection or sterilization treatment. The surface of the tissue is washed with running water and then ethanol. Next, the surface is sterilized with aqueous sodium hypochlorite, optionally with stirring. Thereafter, the surface is washed with sterilized water.

The induction step includes culturing a tissue including a node, axillary bud or apical bud of a rubber tree in an induction medium containing a plant growth hormone and a carbon source to induce and form a shoot. Although the induction medium may be liquid or solid, solid culture is preferred because the shoot can be easily induced by culturing the tissue inserted in the medium. Moreover, in the case where the induction medium is a liquid medium, static culture or shaking culture may be performed.

When tissues subjected to sterilization or disinfection are used, it is preferable to cut off the cut end to remove the effect of the disinfectant or sterilizer before use in culture.

Examples of the plant growth hormone include auxin plant hormones and/or cytokinin plant hormones. Among these, cytokinin plant hormones are preferred.

Examples of auxin plant hormones include 2,4-dichlorophenoxyacetic acid, 1-naphthaleneacetic acid, indole-3-butyric acid, indole-3-acetic acid, indolepropionic acid, chlorophenoxyacetic acid, naphthoxyacetic acid, phenylacetic acid, 2,4,5-trichlorophenoxyacetic acid, p-chlorophenoxyacetic acid, 2-methyl-4-chlorophenoxyacetic acid, 4-fluorophenoxyacetic acid, 2-methoxy-3,6-dichlorobenzoic acid, 2-phenyl acid, picloram, and picolinic acid. Among these, 2,4-dichlorophenoxyacetic acid, 1-naphthaleneacetic acid, and indole-3-butyric acid are preferred, and 2,4-dichlorophenoxyacetic acid or 1-naphthaleneacetic acid is more preferred.

Examples of cytokinin plant hormones include benzyladenine, kinetin, zeatin, benzylaminopurine, isopentenyl aminopurine, thidiazuron, isopentenyl adenine, zeatin riboside, and dihydrozeatin. Among these, benzyladenine, kinetin, and zeatin are preferred, benzyladenine or kinetin is more preferred, and benzyladenine is still more preferred.

The carbon source is not particularly limited, and examples include sugars such as sucrose, glucose, trehalose, fructose, lactose, galactose, xylose, mannitol, sorbitol, xylitol, erythritol, and maltose. Among these, sucrose is preferred.

The induction medium preferably further contains activated carbon to prevent accumulation of growth inhibitors in the tissue. Moreover, the induction medium preferably further contains silver nitrate to promote shoot formation. Furthermore, the induction medium may contain coconut water (coconut milk) to promote shoot formation.

The induction medium may be any of the following base media which are supplemented with the plant growth hormone: basal media such as White's medium (disclosed on pages 20 to 36 of Shokubutsu Saibo Kogaku Nyumon (Introduction to Plant Cell Engineering), Japan Scientific Societies Press), Heller's medium (Heller R, Bot. Biol. Veg. Paris 14, 1-223 (1953)), SH medium (medium of Schenk and Hildebrandt), MS medium (medium of Murashige and Skoog) (disclosed on pages 20 to 36 of Shokubutsu Saibo Kogaku Nyumon (Introduction to Plant Cell Engineering), Japan Scientific Societies Press), LS medium (medium of Linsmaier and Skoog) (disclosed on pages 20 to 36 of Shokubutsu Saibo Kogaku Nyumon (Introduction to Plant Cell Engineering), Japan Scientific Societies Press), Gamborg medium, B5 medium (disclosed on pages 20 to 36 of Shokubutsu Saibo Kogaku Nyumon (Introduction to Plant Cell Engineering), Japan Scientific Societies Press), MB medium, and WP medium (Woody Plant: for woody plants)(the disclosures of the foregoing documents are incorporated by reference herein), as well as modified basal media obtained by modifying the composition of the basal media, and the like. Among these, MS medium, B5 medium, or WP medium, supplemented with the plant growth hormone is preferred, and MS medium or modified MS media obtained by modifying the composition of MS medium, supplemented with the plant growth hormone is more preferred.

To prepare the induction medium as a solid medium, the medium may be made solid using a solidifying agent. Examples of the solidifying agent include, but are not limited to, agar, gellan gum (e.g. Gelrite, Phytagel), and agarose.

The suitable composition and culture conditions of the induction medium vary depending on the type of plant, and also vary depending on whether the medium is a liquid medium or a solid medium. Typically (particularly in the case of rubber trees), the induction medium has the following composition.

The concentration of the carbon source in the induction medium is preferably 0.1 mass % or more, and more preferably 1.0 mass % or more. The concentration of the carbon source is preferably 10 mass % or less, and more preferably 5.0 mass % or less. The concentration of the carbon source as used herein means the concentration of sugars.

It is preferable that substantially no auxin plant hormone be added to the induction medium. Specifically, the concentration of the auxin plant hormone in the induction medium is preferably 1.0 mg/L or less, more preferably 0.1 mg/L or less, still more preferably 0.05 mg/L or less, and particularly preferably 0.01 mg/L or less.

When a cytokinin plant hormone is added to the induction medium, the concentration of the cytokinin plant hormone in the induction medium is preferably 0.01 mg/L or more, more preferably 0.1 mg/L or more, still more preferably 0.5 mg/L or more, and particularly preferably 0.8 mg/L or more. The concentration of the cytokinin plant hormone is preferably 7.0 mg/L or less, and more preferably 6.0 mg/L or less.

In particular, when benzyladenine is used as the cytokinin plant hormone, the concentration of benzyladenine is preferably 4.0 mg/L to 6.0 mg/L, and most preferably 5.0 mg/L. On the other hand, when kinetin is used as the cytokinin plant hormone, the concentration of kinetin is preferably 0.8 mg/L to 1.2 mg/L, and most preferably 1.0 mg/L.

The concentration of activated carbon in the induction medium is preferably 0.01 mass % or more, and more preferably 0.03 mass % or more. The concentration of activated carbon is preferably 1.0 mass % or less, and more preferably 0.1 mass % or less.

The concentration of silver nitrate in the induction medium is preferably 0.1 mg/L or more, more preferably 0.3 mg/L or more, and still more preferably 0.5 mg/L or more. The concentration of silver nitrate is preferably 5.0 mg/L or less, and more preferably 3.0 mg/L or less.

The pH of the induction medium is preferably 4.0 to 10.0, more preferably 5.0 to 6.5, and still more preferably 5.5 to 6.0.

In the present specification, the pH of solid media means the pH of media supplemented with all the components except the solidifying agent.

The induction step is usually carried out in a controlled environment where the culture conditions, including temperature and illumination time, are controlled. The culture conditions may be appropriately chosen. For example, the culture temperature is preferably 0° C. to 40° C., more preferably 20° C. to 40° C., and still more preferably 25° C. to 35° C. The culture may be carried out in a dark place or in a bright place. The light conditions may, for example, be under illumination at 12.5 μmol/m²/s with a light time of 14 to 16 hours. The culture time is not particularly limited, but is preferably 1 to 10 weeks, and more preferably 3 to 5 weeks.

The concentration of the solidifying agent in the induction medium as a solid medium is preferably 0.1 mass % or more, more preferably 0.2 mass % or more, and still more preferably 0.5 mass % or more. The concentration of the solidifying agent is preferably 2.0 mass % or less, more preferably 1.1 mass % or less, and still more preferably 0.8 mass % or less.

Particularly preferred among the above-described conditions is such that the plant growth hormone is a cytokinin plant hormone, and especially benzyladenine or kinetin, at a concentration of 0.8 mg/L to 6.0 mg/L, and the culture temperature is 25° C. to 35° C.

As described above, it is possible to induce and form a shoot by culturing a tissue including a node, axillary bud, or apical bud of a rubber tree in the above-described induction medium.

By subjecting the shoot formed through the induction step to a propagation step described later, it is possible to increase the number of shoots to mass-propagate a tissue that can be used as a graft. The shoot formed through the induction step can be subjected to the propagation step as long as stable growth of the shoot is confirmed. However, for example, if the shoot is cultured in the induction medium for 4 weeks, the shoot can not only be induced but also be elongated, and thus not only the induced shoot, but also such an induced and elongated shoot may be subjected to the propagation step. Here, the degree of elongation of the shoot may be appropriately adjusted according to the culture conditions such as culture time in the induction medium. Furthermore, the shoot induced through the induction step may be subjected to an elongation step described later to elongate the shoot before the propagation step.

### (Elongation Step)

The elongation step includes culturing the shoot formed through the induction step in an elongation medium containing a plant growth hormone and a carbon source to elongate the shoot. Specifically, it may include inserting and transplanting the shoot (of, for example, about 2 cm) formed through the induction step into the elongation medium and culturing it for about 4 weeks to elongate the shoot and, further, obtain a new bud.

Although the elongation medium may be liquid or solid, solid culture is preferred because the shoot can be easily elongated by culturing the tissue inserted in the medium. Moreover, in the case where the elongation medium is a liquid medium, static culture or shaking culture may be performed.

The elongation medium contains a plant growth hormone and a carbon source, and examples of the plant growth hormone include auxin plant hormones and/or cytokinin plant hormones. Among these, combinations of auxin plant hormones and cytokinin plant hormones are preferred.

The auxin plant hormone may be as mentioned for the auxin plant hormone used in the induction medium. In particular, 2,4-dichlorophenoxyacetic acid, 1-naphthaleneacetic acid, and indole-3-butyric acid are preferred, 2,4-dichlorophenoxyacetic acid or 1-naphthaleneacetic acid is more preferred, and 1-naphthaleneacetic acid is particularly preferred.

The cytokinin plant hormone may be as mentioned for the cytokinin plant hormone used in the induction medium. In particular, benzyladenine, kinetin, and zeatin are preferred, benzyladenine or kinetin is more preferred, and benzyladenine is still more preferred.

The carbon source used in the elongation medium is not particularly limited, and may be as mentioned for the carbon source used in the induction medium. In particular, sucrose is preferred.

Similarly to the induction medium, the elongation medium preferably further contains activated carbon or silver nitrate.

The elongation medium may be as mentioned for the base medium (e.g., basal media or modified basal media obtained by modifying the composition of the basal media) supplemented with the plant growth hormone used as the induction medium. In particular, MS medium, B5 medium, or WP medium, supplemented with the plant growth hormone is preferred, and MS medium or modified MS media obtained by modifying the composition of MS medium, supplemented with the plant growth hormone is more preferred.

To prepare the elongation medium as a solid medium, the medium may be made solid using a solidifying agent. Examples of the solidifying agent include, but are not limited to, agar, gellan gum (e.g. Gelrite, Phytagel), and agarose.

The suitable composition and culture conditions of the elongation medium vary depending on the type of plant, and also vary depending on whether the medium is a liquid medium or a solid medium. Typically (particularly in the case of rubber trees), the elongation medium has the following composition.

The concentration of the carbon source in the elongation medium is preferably 0.1 mass % or more, and more preferably 1.0 mass % or more. The concentration of the carbon source is preferably 10 mass % or less, and more preferably 5.0 mass % or less.

When an auxin plant hormone is added to the elongation medium, the concentration of the auxin plant hormone in the elongation medium is preferably 0.01 mg/L or more, more preferably 0.03 mg/L or more, and still more preferably 0.05 mg/L or more. The concentration of the auxin plant hormone is preferably 2.0 mg/L or less, more preferably 1.0 mg/L or less, still more preferably 0.1 mg/L or less, and particularly preferably 0.08 mg/L or less.

When a cytokinin plant hormone is added to the elongation medium, the concentration of the cytokinin plant hormone in the elongation medium is preferably 0.01 mg/L or more, more preferably 0.1 mg/L or more, still more preferably 0.5 mg/L or more, and particularly preferably 0.8 mg/L or more. The concentration of the cytokinin plant hormone is preferably 5.0 mg/L or less, and more preferably 2.0 mg/L or less.

The concentration of activated carbon in the elongation medium is preferably 0.01 mass % or more, and more preferably 0.03 mass % or more. The concentration of activated carbon is preferably 1.0 mass % or less, and more preferably 0.1 mass % or less.

The concentration of silver nitrate in the elongation medium is preferably 0.1 mg/L or more, more preferably 0.3 mg/L or more, and still more preferably 0.5 mg/L or more. The concentration of silver nitrate is preferably 5.0 mg/L or less, and more preferably 3.0 mg/L or less.

The pH of the elongation medium is preferably 4.0 to 10.0, more preferably 5.0 to 6.5, and still more preferably 5.5 to 6.0.

The elongation step is usually carried out in a controlled environment where the culture conditions, including temperature and illumination time, are controlled. The culture conditions may be appropriately chosen. For example, the culture temperature is preferably 0° C. to 40° C., more preferably 20° C. to 40° C., and still more preferably 25° C. to 35° C. The culture may be carried out in a dark place or in a bright place. The light conditions may, for example, be under illumination at 12.5 μmol/m²/s with a light time of 14 to 16 hours. The culture time is not particularly limited, but is preferably 1 to 10 weeks, and more preferably 3 to 5 weeks.

The concentration of the solidifying agent in the elongation medium as a solid medium is preferably 0.1 mass % or more, more preferably 0.2 mass % or more, and still more preferably 0.5 mass % or more. The concentration of the solidifying agent is preferably 2.0 mass % or less, more preferably 1.1 mass % or less, and still more preferably 0.8 mass % or less.

Particularly preferred among the above-described conditions is such that the plant growth hormone is a combination of an auxin plant hormone, especially 1-naphthaleneacetic acid, and a cytokinin plant hormone, especially benzyladenine, at concentrations of 0.05 to 0.08 mg/L and 0.8 to 2.0 mg/L, respectively, and the culture temperature is 25° C. to 35° C.

The elongation medium may preferably have a layered structure including multiple separate layers of which at least one layer is formed of a solid medium and at least one layer is formed of a liquid medium. With the elongation medium having such a structure, it is possible to further promote elongation of the shoot.

In the case of the elongation medium having the above-described layered structure, the composition of the elongation medium as a whole satisfies the preferred ranges described above.

The elongation medium having the layered structure is more preferably an embodiment in which the upper layer is a liquid layer and the lower layer is a solid layer, and still more preferably an embodiment having two layers including one liquid upper layer and one solid lower layer. A specific example may be the following embodiment:

The upper layer is a liquid layer obtained by adding an auxin plant hormone and a cytokinin plant hormone to ½ MS medium and the lower layer is a solid layer obtained by adding a cytokinin plant hormone, silver nitrate, a carbon source, activated carbon, and a solidifying agent to MS medium.

In the elongation medium having the two-layered structure, the amount of the cytokinin plant hormone added is preferably 0.01 mg/L to 0.05 mg/L for the liquid layer and 0.8 mg/L to 2.0 mg/L for the solid layer. On the other hand, the amount of the auxin plant hormone added is preferably 0.03 mg/L to 0.1 mg/L for the liquid layer and 0.01 mg/L or less (substantially zero) for the solid layer. The amount of silver nitrate added is preferably 0.01 mg/L or less (substantially zero) for the liquid layer and 0.5 mg/L to 2.5 mg/L for the solid layer. Moreover, the amount of activated carbon added is preferably 0.001 mass % or less (substantially zero) for the liquid layer and 0.03 mass % to 0.1 mass % for the solid layer.

As described above, it is possible to elongate the shoot by culturing the shoot formed through the induction step in the above-described elongation medium. Moreover, the elongation step not only elongates the shoot, but also forms a new shoot. The shoot elongated through the elongation step is used in a propagation step which will be described below.

### (Propagation Step)

The propagation step includes collecting the shoot formed through the induction step or the shoot elongated through the elongation step, dividing the shoot into multiple shoots, and then culturing the divided shoot in an induction medium containing a plant growth hormone and a carbon source to form a shoot. With this step, it is possible to increase the number of shoots to mass-propagate a tissue that can be used as a graft. Further, it is possible to further mass-propagate the tissue by repeating the step or by subculture.

In the propagation step, if the shoot formed through the induction step or the shoot elongated through the elongation step is collected without collecting the node, axillary bud, and apical bud portions, the number of shoots can be stably increased in the propagation step, which is preferred.

The collected shoot can be divided by conventionally known methods and the size of the shoot divided may be appropriately chosen.

The induction medium used in the propagation step may be as mentioned for the induction medium used in the induction step.

As described above, the propagation step includes collecting the shoot, dividing it into multiple shoots, and then culturing the divided shoot in the above-described induction medium to form a shoot, thereby enabling mass propagation of the shoot. The shoot formed through the propagation step is used in a rooting step which will be described later. The shoot can be subjected to the rooting step as long as stable growth of the shoot is confirmed. However, the formed shoot may be subjected to the rooting step after it is subcultured and grown in the induction medium, or the shoot may be subjected to the above-described elongation step before the rooting step.

### (Rooting Step)

The rooting step includes culturing the shoot in a root induction medium to root the shoot. The shoot used in this step is the shoot formed through the propagation step, which may be elongated through the elongation step and then used, or the shoot induced and formed through the induction step may directly be used.

In the rooting step, for example, the shoot formed through the propagation step is cultured in a root induction medium to root the shoot. Although the root induction medium may be liquid or solid, solid culture is preferred because the shoot can be easily rooted by culturing the shoot inserted in the medium. Moreover, in the case where the root induction medium is a liquid medium, static culture or shaking culture may be performed.

The root induction medium contains a plant growth hormone and a carbon source, and examples of the plant growth hormone include auxin plant hormones and/or cytokinin plant hormones. Among these, auxin plant hormones are preferred.

The auxin plant hormone may be as mentioned for the auxin plant hormone used in the induction medium. In particular, 2,4-dichlorophenoxyacetic acid, 1-naphthaleneacetic acid, indole-3-butyric acid, and indole-3-acetic acid are preferred, and indole-3-butyric acid is more preferred.

The cytokinin plant hormone may be as mentioned for the cytokinin plant hormone used in the induction medium. In particular, benzyladenine, kinetin, and zeatin are preferred, and benzyladenine or kinetin is more preferred.

The carbon source used in the root induction medium is not particularly limited, and may be as mentioned for the carbon source used in the induction medium. In particular, sucrose is preferred.

Similarly to the induction medium, the root induction medium preferably further contains activated carbon or silver nitrate.

The root induction medium may be as mentioned for the base medium (e.g., basal media or modified basal media obtained by modifying the composition of the basal media) supplemented with the plant growth hormone used as the induction medium. In particular, MS medium, B5 medium, or WP medium, supplemented with the plant growth hormone is preferred, and MS medium or modified MS media obtained by modifying the composition of MS medium, supplemented with the plant growth hormone is more preferred.

To prepare the root induction medium as a solid medium, the medium may be made solid using a solidifying agent. Examples of the solidifying agent include, but are not limited to, agar, gellan gum (e.g. Gelrite, Phytagel), and agarose.

The suitable composition and culture conditions of the root induction medium vary depending on the type of plant, and also vary depending on whether the medium is a liquid medium or a solid medium. Typically (particularly in the case of rubber trees), the root induction medium has the following composition.

The concentration of the carbon source in the root induction medium is preferably 0.1 mass % or more, and more preferably 1.0 mass % or more. The concentration of the carbon source is preferably 10 mass % or less, and more preferably 5.0 mass % or less.

When an auxin plant hormone is added to the root induction medium, the concentration of the auxin plant hormone in the root induction medium is preferably 0.5 mg/L or more, more preferably 1.0 mg/L or more, and still more preferably 3.0 mg/L or more. The concentration of the auxin plant hormone is preferably 10 mg/L or less, more preferably 6.0 mg/L or less, and still more preferably 5.0 mg/L or less.

It is preferable that substantially no cytokinin plant hormone be added to the root induction medium. Specifically, the concentration is preferably 1.0 mg/L or less, more preferably 0.1 mg/L or less, still more preferably 0.05 mg/L or less, and particularly preferably 0.01 mg/L or less.

The concentration of activated carbon in the root induction medium is preferably 0.01 mass % or more, and more preferably 0.03 mass % or more. The concentration of activated carbon is preferably 1.0 mass % or less, and more preferably 0.1 mass % or less.

The concentration of silver nitrate in the root induction medium is preferably 0.1 mg/L or more, more preferably 0.3 mg/L or more, and still more preferably 0.5 mg/L or more. The concentration of silver nitrate is preferably 5.0 mg/L or less, and more preferably 3.0 mg/L or less.

The pH of the root induction medium is preferably 4.0 to 10.0, more preferably 5.0 to 6.5, and still more preferably 5.5 to 6.0.

The rooting step is usually carried out in a controlled environment where the culture conditions, including temperature and illumination time, are controlled. The culture conditions may be appropriately chosen. For example, the culture temperature is preferably 0° C. to 40° C., more preferably 20° C. to 40° C., and still more preferably 25° C. to 35° C. The culture may be carried out in a dark place or in a bright place. The light conditions may, for example, be under illumination at 12.5 μmol/m²/s with a light time of 14 to 16 hours. The culture time is not particularly limited, but is preferably 1 to 10 weeks, and more preferably 4 to 8 weeks.

The concentration of the solidifying agent in the root induction medium as a solid medium is preferably 0.1 mass % or more, more preferably 0.2 mass % or more, and still more preferably 0.5 mass % or more. The concentration of the solidifying agent is preferably 2.0 mass % or less, more preferably 1.1 mass % or less, and still more preferably 0.8 mass % or less.

Particularly preferred among the above-described conditions is such that the plant growth hormone is an auxin plant hormone, and especially indole-3-butyric acid, at a concentration of 3.0 mg/L to 6.0 mg/L, and the culture temperature is 25° C. to 35° C.

As described above, by culturing the shoot in the above-described root induction medium, it is possible to root the shoot, whereby rooted shoots (in the present specification, the rooted shoot is also referred to as “young plant”) can be obtained and therefore clone seedlings, which are complete plants, can be formed. The young plant may be directly transplanted into soil, but it is preferably subjected to an acclimatization step which will be described later before transplanting into soil.

It is also possible to mass-produce superior clone seedlings by repeatedly implementing the regeneration method or the propagation method of the present invention using the clone seedlings formed as described above.

### (Acclimatization Step)

The acclimatization step includes treating the shoot rooted through the rooting step with a disinfectant, followed by transplanting the shoot into a cultivation soil and acclimatizing the transplanted shoot.

Aseptically cultured plantlets and young plants obtained by tissue-culturing tissue pieces or culture cells of plants, and young plants obtained by aseptic culture from seeds are aseptically grown in an environment where illumination and temperature are controlled, and thus the plants are held in an environment where they are sufficiently protected. When such plants are used, they are preferably subjected to an acclimatization step to release them from the controlled environment and grow them in a natural environment.

In acclimatization which involves changes in the environment or artificial manipulations such as transplant, plants may be damaged. Moreover, since young plants obtained by tissue culture or the like are grown in agar media; they may have a small number of roots so that the rhizomes can grow poorly.

For this reason, some young plants obtained by aseptic culture cannot take root sufficiently when transplanted directly into normal soil. Thus, a problem in efficiently carrying out mass propagation of seeds and plantlets by tissue culture may be caused in some cases.

The conventional acclimatization methods include a method of opening a aseptic culture vessel to take out a young plant aseptically cultured therein, and washing away the medium such as agar attached to the roots, followed by transferring the young plant to an environment for acclimatization, where the young plant is planted into a culture soil, exposed to for example an environment that is less contaminated with bacteria to acclimatize the young plant to the air, and then monitored.

In contrast, in the acclimatization step used in the present invention, the shoot (young plant) rooted through the rooting step is treated with a disinfectant and then transplanted into a cultivation soil. Thus, it is possible to efficiently and stably perform acclimatization without requiring complicated controls such as aseptic operations or controls according to the growth stage. It is also possible to reduce death or growth arrest after transplanting, thereby efficiently acclimatizing the young plant.

In the acclimatization step, first, the root induction medium used in the rooting step is carefully removed from the young plant, and the medium attached to the young plant is removed for example by gently washing away the medium with running water. Thereafter, the young plant is treated with a disinfectant. The treatment with a disinfectant may be carried out by, for example, a method of immersing the roots of the young plant in a solution containing a disinfectant.

Examples of the disinfectant include, but are not limited to, aldehyde disinfectants, chlorinated disinfectants, oxidants, iodine agents, alcohol disinfectants, biguanide disinfectants, quaternary ammonium salts, amphoteric surfactants, and dye disinfectants. Among these, iodine disinfectants such as povidone-iodine and tincture of iodine are preferred, and povidone-iodine is particularly preferred.

The concentration of the disinfectant used may be appropriately selected as long as it is not less than a concentration where the effect is produced on the particular plant. For rubber trees, the concentration is preferably 0.001 mass % or more, and more preferably 0.005 mass % or more. Also, the concentration is preferably 0.1 mass % or less, and more preferably 0.05 mass % or less.

Moreover, the duration of immersion of the young plant in the solution containing the disinfectant may also be appropriately selected for sufficient disinfection.

After the treatment with the disinfectant, the young plant is transplanted into a cultivation soil. The cultivation soil may be either an organic soil or an inorganic soil, or may include both organic and inorganic soils. Specific examples include inorganic materials such as attapulgite, montmorillonite, zeolite, Akadama soil, Kanuma soil, andosol, fired Akadama soil, vermiculite, perlite, and fossil shellfish; organic materials such as peat moss, charcoal, pulp, straw, bagasse, oil cakes, fish cakes, bone meal, blood meal, and crab shells; and mixtures thereof.

The young plant transplanted in the cultivation soil is cultured, for example, under illumination for 10 hours or more while the upper portion of the young plant is covered with a plastic container, plastic bag, or the like to maintain the humidity. Then, after the young plant has been acclimatized to the cultivation soil environment, the young plant is gradually exposed to a natural environment, for example, by gradually removing the cover from the upper portion. The acclimatization is completed when the young plant is placed in the natural environment, for example, when the cover is completely removed.

The material of the cover is not limited to plastics. The material is not particularly limited and may be appropriately selected as long as it is a material that is light-permeable and can maintain the humidity inside the cover.

The light conditions in the culture may, for example, be under illumination at 12.5 μmol/m²/s with a light time of 14 to 16 hours.

The culture time until the young plant has been acclimatized to the cultivation soil environment is not particularly limited, but is, for example, preferably 1 to 10 weeks, and more preferably 1 to 4 weeks.

Thus, in another suitable embodiment of the present invention, the acclimatization step includes, after transplanting the rooted shoot (young plant) treated with the disinfectant into the cultivation soil, gradually exposing the transplanted shoot to a natural environment to adapt the shoot to the environment for acclimatization. The gradual exposure to a natural environment may be carried out by, for example, a method of gradually removing the cover covering the upper portion of the young plant while checking the state of the young plant after the young plant has been acclimatized to the cultivation soil environment; or a method of opening, after the young plant has been acclimatized to the cultivation soil environment, a portion of the cover covering the upper portion of the young plant to make the environment of the young plant continuous with the outside natural environment, shifting the position of the young plant inside the cover while checking the state of the young plant, thereby gradually bringing the young plant closer to the opening of the cover.

As described above, in the present invention, it is possible to induce a shoot from a tissue including a node, axillary bud, or apical bud of a rubber tree, thereby stably forming the shoot, and to elongate, root, and acclimatize the formed shoot, thereby stably regenerating the rubber tree. Moreover, it is possible to stably propagate clone seedlings of the rubber tree by tissue-culture and propagation in a controlled environment.

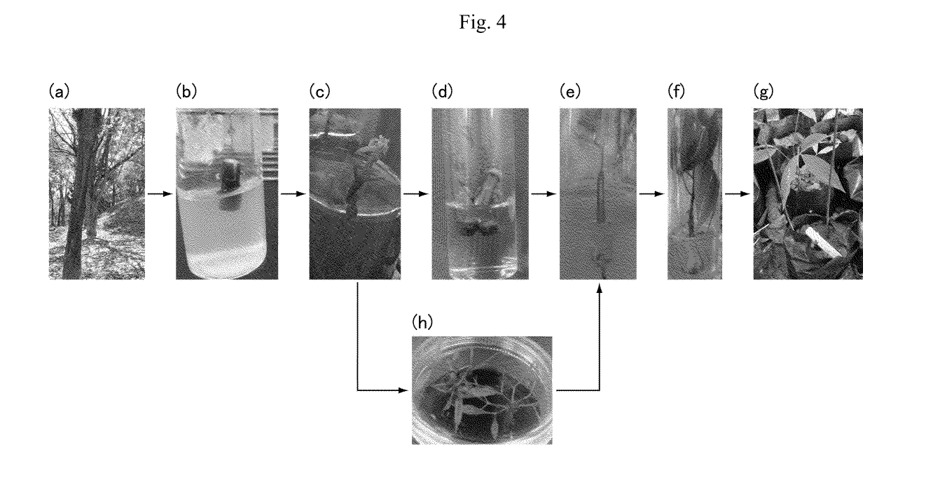

A flowchart illustrating the flow of a series of steps of examples of methods of regenerating or propagating a rubber tree of the present invention is shown in FIG. 4 as a summary. First, in an example of a method of regenerating a rubber tree of the present invention, a tissue including an axillary bud is collected from a mature tree (a) of Hevea brasiliensis and cultured in an induction medium (b) to form a shoot (c) (induction step); the formed shoot is cultured in an elongation medium (d) (elongation step); the elongated shoot is cultured in a root induction medium to root and grow the shoot (e, f) (rooting step); and then the shoot is acclimatized (g) (acclimatization step). Moreover, in an example of a method of propagating a rubber tree of the present invention, a tissue including an axillary bud is collected from a mature tree (a) of Hevea brasiliensis and cultured in an induction medium (b) to form a shoot (c) (induction step); the formed shoot is collected and divided into multiple shoots, and then the divided shoot is cultured in an induction medium to form a shoot (h) (propagation step); the formed shoot is cultured in a root induction medium to root and grow the shoot (e, f) (rooting step); and then the shoot is acclimatized (g) (acclimatization step).

Although the elongation step in the above case is applied to the shoot formed through the induction step, the elongation method used in the elongation step can be applied not only to the shoot formed through the induction step but also to shoots formed by processes other than the induction step to elongate them as long as they are shoots of rubber trees. Thus, another aspect of the present invention is a method of elongating a shoot of a rubber tree which includes culturing a shoot of a rubber tree in an elongation medium containing a plant growth hormone and a carbon source to elongate the shoot.

Moreover, although the above rooting step is applied to the shoot formed through the induction step, the shoot elongated through the elongation step, and the shoot formed through the propagation step, the rooting method used in the rooting step can be applied not only to the shoot formed through the induction step, the shoot elongated through the elongation step, and the shoot formed through the propagation step, but also to shoots formed by processes other than the induction step, the elongation step, and the propagation step to root them as long as they are shoots of rubber trees. Thus, another aspect of the present invention is a method of rooting a shoot of a rubber tree which includes culturing a shoot of a rubber tree in a root induction medium containing a plant growth hormone and a carbon source to root the shoot.

The shoots of rubber trees that can be used in the elongation method and rooting method are not particularly limited, and shoots formed by any process can be used.

Moreover, although the above acclimatization step is applied to the shoot rooted through the rooting step, the acclimatization method used in the acclimatization step can be applied not only to the shoot rooted through the rooting step but also to other young shoots having rhizomes that can be used as materials of young plants of rubber trees, to acclimatize them. Thus, another aspect of the present invention is an acclimatization method of treating a young rubber tree with a disinfectant, followed by transplanting the treated young rubber tree in a cultivation soil and acclimatizing the transplanted young rubber tree.

The young shoots having rhizomes that can be used as materials of young plants include those which have been subcultured and maintained by conventional tissue culture. Besides, plants prepared by redifferentiation from undifferentiated tissues may be used. Furthermore, aseptic seedlings obtained by embryo culture using seeds may also be used.

The aseptic young plants or seedlings may be prepared by any method, and conventionally known methods may be used. For example, a tissue piece or the like aseptically cut from a stem tip, leaf, flower, root, stem, seed, or the like of a parent plant is placed on a culture medium containing nutrients for growing plants, and the resulting callus, shoot primordium, precocious branch or the like is transplanted into a propagation medium to propagate the cellular tissue, thereby obtaining an adventitious bud or the like. The obtained tissue is transplanted into a rooting medium to root and elongate the bud, thereby obtaining a cultured plant (young plant). Moreover, in another possible method of preparing a cultured plant (young plant), a growing point is cut from a parent plant and then placed in a medium to root and elongate a bud.

### EXAMPLES

The present invention will be described in detail with reference to Examples, but the present invention is not limited to the examples.

The chemicals used in Examples are listed below.

### BA: Benzyladenine

### KI: Kinetin

NAA: 1-naphthaleneacetic acid
IBA: Indole-3-butyric acid
Silver Nitrate: Silver nitrate made by Merck
Coconut Water: Commercially available coconut palm fruit
Gellant: Agar (powder) made by Fluka
Disinfectant: Pyrad-Povidone (trade name) (povidone-iodine solution) made by The United Drug
H. brasiliensis: Hevea brasiliensis that naturally grew within Prince of Songkla University

### Examples 1 to 18, Comparative Examples 1 to 6

### Induction Step

A tissue including a node, axillary bud, or apical bud was collected from a mature tree or a plantlet of H. brasiliensis. Moreover, a tissue including a node, axillary bud, or apical bud was collected from a seedling (aseptic seedling) obtained by aseptically germinating and culturing a seed of H. brasiliensis in vitro.

Next, the tissue including a node, axillary bud, or apical bud collected from the mature tree or plantlet was washed with running water and further washed with 70 mass % ethanol, followed by sterilization with aqueous sodium hypochlorite diluted to about 5 to 10 volume %, and then washing with sterilized water.

Next, the sterilized tissue or the tissue derived from the aseptic seedling was inserted into an induction medium (solid medium) and cultured (induction step). The induction medium was prepared in such a manner that a plant hormone, silver nitrate, sucrose, activated carbon, and coconut water at predetermined concentrations shown in the Table 1 below were added to MS medium (disclosed on pages 20 to 36 of Shokubutsu Saibo Kogaku Nyumon (Introduction to Plant Cell Engineering), Japan Scientific Societies Press), the pH of the medium was adjusted to 5.7, and then a gellant was added at 0.75 mass %, followed by autoclave sterilization (at 121° C. for 20 minutes) and then cooling in a clean bench.

The tissue of H. brasiliensis was inserted into the induction medium (solid medium) and then cultured for 4 weeks at a culture temperature of 28° C. under illumination at 12.5 μmol/m²/s with a light time of 16 hours to induce a shoot. The degree of shoot induction was evaluated based on the following rating criteria.

### [Ratings of Shoot Induction Rate]

A shoot induction rate was calculated using the following equation and the calculated shoot induction rate was evaluated based on the following criteria.

(shoot induction rate (%))={(the number of tissues in which shoot formation was confirmed)/(the number of tissues transplanted into the induction medium)}×100

### Rating Criteria:

A: Shoot induction rate was 75% or more;
B: Shoot induction rate was at least 50% but less than 75%;
C: Shoot induction rate was less than 25%.

The tissue, the composition of the medium, the culture temperature, and the rating of shoot induction rate in Examples 1 to 18 and Comparative Examples 1 to 6 are shown in Table 1.

TABLE 1

| | | | | Conc. of | | Conc. of | Conc. of | | | Rating of |
|---|---|---|---|---|---|---|---|---|---|---|
| | | | | silver | Sucrose | activated | coconut | Gellant | Culture | shoot |
| | | BA conc. | KI conc. | nitrate | conc. | carbon | water | conc. | temperature | induction |
| | Tissue | (mg/L) | (mg/L) | (mg/L) | (mass %) | (mass %) | (mass %) | (mass %) | (° C.) | rate |
| Ex. 1 | Node of mature | 5.0 | — | 1.0 | 3.0 | 0.05 | — | 0.75 | 28 | A |
| | tree of | | | | | | | | | |
| | H. brasiliensis | | | | | | | | | |
| Ex. 2 | Node of | 5.0 | — | 1.0 | 3.0 | 0.05 | — | 0.75 | 28 | A |
| | plantlet of | | | | | | | | | |
| | H. brasiliensis | | | | | | | | | |
| Ex. 3 | Node of aseptic | 5.0 | — | 1.0 | 3.0 | 0.05 | — | 0.75 | 28 | A |
| | seedling of | | | | | | | | | |
| | H. brasiliensis | | | | | | | | | |
| Ex. 4 | Axillary bud of | 5.0 | — | 1.0 | 3.0 | 0.05 | — | 0.75 | 28 | A |
| | mature tree of | | | | | | | | | |
| | H. brasiliensis | | | | | | | | | |
| Ex. 5 | Axillary bud of | 5.0 | — | 1.0 | 3.0 | 0.05 | — | 0.75 | 28 | A |
| | plantlet of | | | | | | | | | |
| | H. brasiliensis | | | | | | | | | |
| Ex. 6 | Axillary bud of | 5.0 | — | 1.0 | 3.0 | 0.05 | — | 0.75 | 28 | A |
| | aseptic | | | | | | | | | |
| | seedling of | | | | | | | | | |
| | H. brasiliensis | | | | | | | | | |
| Ex. 7 | Apical bud of | 5.0 | — | 1.0 | 3.0 | 0.05 | — | 0.75 | 28 | A |
| | mature tree of | | | | | | | | | |
| | H. brasiliensis | | | | | | | | | |
| Ex. 8 | Apical bud of | 5.0 | — | 1.0 | 3.0 | 0.05 | — | 0.75 | 28 | A |
| | plantlet of | | | | | | | | | |
| | H. brasiliensis | | | | | | | | | |
| Ex. 9 | Apical bud of | 5.0 | — | 1.0 | 3.0 | 0.05 | — | 0.75 | 28 | A |
| | aseptic | | | | | | | | | |
| | seedling of | | | | | | | | | |
| | H. brasiliensis | | | | | | | | | |
| Ex. | Node of mature | — | 1.0 | 1.0 | 3.0 | 0.05 | 5.0 | 0.75 | 28 | A |
| 10 | tree of | | | | | | | | | |
| | H. brasiliensis | | | | | | | | | |
| Ex. | Node of | — | 1.0 | 1.0 | 3.0 | 0.05 | 5.0 | 0.75 | 28 | A |
| 11 | plantlet of | | | | | | | | | |
| | H. brasiliensis | | | | | | | | | |
| Ex. | Node of aseptic | — | 1.0 | 1.0 | 3.0 | 0.05 | 5.0 | 0.75 | 28 | A |
| 12 | plantlet of | | | | | | | | | |
| | H. brasiliensis | | | | | | | | | |
| Ex. | Axillary bud of | — | 1.0 | 1.0 | 3.0 | 0.05 | 5.0 | 0.75 | 28 | A |
| 13 | mature tree of | | | | | | | | | |
| | H. brasiliensis | | | | | | | | | |
| Ex. | Axillary bud of | — | 1.0 | 1.0 | 3.0 | 0.05 | 5.0 | 0.75 | 28 | A |
| 14 | plantlet of | | | | | | | | | |
| | H. brasiliensis | | | | | | | | | |
| Ex. | Axillary bud of | — | 1.0 | 1.0 | 3.0 | 0.05 | 5.0 | 0.75 | 28 | A |
| 15 | aseptic | | | | | | | | | |
| | seedling of | | | | | | | | | |
| | H. brasiliensis | | | | | | | | | |
| Ex. | Apical bud of | — | 1.0 | 1.0 | 3.0 | 0.05 | 5.0 | 0.75 | 28 | A |
| 16 | mature tree of | | | | | | | | | |
| | H. brasiliensis | | | | | | | | | |
| Ex. | Apical bud of | — | 1.0 | 1.0 | 3.0 | 0.05 | 5.0 | 0.75 | 28 | A |
| 17 | plantlet of | | | | | | | | | |
| | H. brasiliensis | | | | | | | | | |
| Ex. | Apical bud of | — | 1.0 | 1.0 | 3.0 | 0.05 | 5.0 | 0.75 | 28 | A |
| 18 | aseptic | | | | | | | | | |
| | seedling of H. brasiliensis | | | | | | | | | |
| Com. | Node of mature | — | — | — | 3.0 | — | — | 0.75 | 28 | C |
| Ex. 1 | tree of | | | | | | | | | |
| | H. brasiliensis | | | | | | | | | |
| Com. | Node of | — | — | — | 3.0 | — | — | 0.75 | 28 | C |
| Ex. 2 | plantlet of | | | | | | | | | |
| | H. brasiliensis | | | | | | | | | |
| Com. | Axillary bud of | — | — | — | 3.0 | — | — | 0.75 | 28 | C |
| Ex. 3 | mature tree of | | | | | | | | | |
| | H. brasiliensis | | | | | | | | | |
| Com. | Axillary bud of | — | — | — | 3.0 | — | — | 0.75 | 28 | C |
| Ex. 4 | plantlet of | | | | | | | | | |
| | H. brasiliensis | | | | | | | | | |
| Com. | Apical bud of | — | — | — | 3.0 | — | — | 0.75 | 28 | C |
| Ex. 5 | mature tree of | | | | | | | | | |
| | H. brasiliensis | | | | | | | | | |
| Com. | Apical bud of | — | — | — | 3.0 | — | — | 0.75 | 28 | C |
| Ex. 6 | plantlet of | | | | | | | | | |
| | H. brasiliensis | | | | | | | | | |

Shoots were induced well and shoot or multiple bud formation was observed after culture in all Examples 1 to 18. The formed shoots and multiple buds were subcultured by transplanting them into an induction medium having the identical composition every 4 weeks. In contrast, in all Comparative Examples 1 to 6 in which culture was carried out in induction media not supplemented with the cytokinin plant hormone, silver nitrate, and activated carbon, shoot induction was not observed and the tissues were exhausted by continuing the culture.

### <Propagation Step>

The shoots induced by culture in Examples 1 to 18 and then grown to about 3 cm by subculture were each divided with the node, axillary bud, and apical bud portions being left therein, and then transplanted into an induction medium having the identical composition and cultured. As a result, new shoots and multiple buds were further generated from the divided shoots, whereby they could be propagated. By repeating the propagation step, the shoots could be mass-propagated well.

### Examples 19 to 27, Comparative Examples 7 to 15

### Elongation Step

The shoots induced by culture in Examples 1 to 9 and then grown by subculture were each cultured in an elongation medium (elongation step). The elongation medium had a two-layered structure and the two layers were based on MS medium (disclosed on pages 20 to 36 of Shokubutsu Saibo Kogaku Nyumon (Introduction to Plant Cell Engineering), Japan Scientific Societies Press). The lower layer medium was prepared in such a manner that a plant hormone, silver nitrate, sucrose, and activated carbon at predetermined concentrations shown in the Table 2 below were added to MS medium, the pH of the medium was adjusted to 5.7, and a gellant was added at 0.75 mass %, followed by autoclave sterilization (at 121° C. for 20 minutes) and then cooling in a clean bench. The upper layer medium was prepared in such a manner that a plant hormone at a predetermined concentration shown in the Table 2 below was added to ½ MS medium and the pH of the medium was adjusted to 5.7, followed by autoclave sterilization (at 121° C. for 20 minutes) and then cooling in a clean bench. The upper layer medium as a liquid layer was put on the lower layer medium as a solid layer to prepare an elongation medium.

The shoots induced by culture in Examples 1 to 9 and then grown by subculture were each inserted into the lower layer medium (solid layer) of the elongation medium and then cultured for 4 weeks at a culture temperature of 28° C. under illumination at 12.5 μmol/m²/s with a light time of 16 hours. The degree of shoot elongation was evaluated based on the following rating criteria.

### [Ratings of Shoot Elongation Rate]

A shoot elongation rate was calculated using the following equation and the calculated shoot elongation rate was evaluated based on the following rating criteria.

(shoot elongation rate (%))={(the number of shoots in which a new bud was confirmed)/(the number of shoots transplanted in the elongation step)}×100

### Rating Criteria:

A: Shoot elongation rate was 70% or more;
C: Shoot elongation rate was less than 70%.

The shoot, the composition of the medium, the culture temperature, and the rating of shoot elongation rate in Examples 19 to 27 and Comparative Examples 7 to 15 are shown in Table 2.

TABLE 2

| | | | | | | Conc. of | | Conc. of | | | Rating of |
|---|---|---|---|---|---|---|---|---|---|---|---|
| | | | | | | silver | Sucrose | activated | Gellant | Culture | shoot |
| | | | | BA conc. | NAA conc. | nitrate | conc. | carbon | conc. | temperature | elongation |
| | Shoot | Layer | MS medium | (mg/L) | (mg/L) | (mg/L) | (mass %) | (mass %) | (mass %) | (° C.) | rate |
| Ex. | Ex. 1 | Upper layer | Amount: ½ | 0.03 | 0.06 | — | — | — | — | 28 | A |
| 19 | | Lower layer | Amount: 1 | 1.0 | — | 1.0 | 3.0 | 0.05 | 0.75 | | |
| Ex. | Ex. 2 | Upper layer | Amount: ½ | 0.03 | 0.06 | — | — | — | — | 28 | A |
| 20 | | Lower layer | Amount: 1 | 1.0 | — | 1.0 | 3.0 | 0.05 | 0.75 | | |
| Ex. | Ex. 3 | Upper layer | Amount: ½ | 0.03 | 0.06 | — | — | — | — | 28 | A |
| 21 | | Lower layer | Amount: 1 | 1.0 | — | 1.0 | 3.0 | 0.05 | 0.75 | | |
| Ex. | Ex. 4 | Upper layer | Amount: ½ | 0.03 | 0.06 | — | — | — | — | 28 | A |
| 22 | | Lower layer | Amount: 1 | 1.0 | — | 1.0 | 3.0 | 0.05 | 0.75 | | |
| Ex. | Ex. 5 | Upper layer | Amount: ½ | 0.03 | 0.06 | — | — | — | — | 28 | A |
| 23 | | Lower layer | Amount: 1 | 1.0 | — | 1.0 | 3.0 | 0.05 | 0.75 | | |
| Ex. | Ex. 6 | Upper layer | Amount: ½ | 0.03 | 0.06 | — | — | — | — | 28 | A |
| 24 | | Lower layer | Amount: 1 | 1.0 | — | 1.0 | 3.0 | 0.05 | 0.75 | | |
| Ex. | Ex. 7 | Upper layer | Amount: ½ | 0.03 | 0.06 | — | — | — | — | 28 | A |
| 25 | | Lower layer | Amount: 1 | 1.0 | — | 1.0 | 3.0 | 0.05 | 0.75 | | |
| Ex. | Ex. 8 | Upper layer | Amount: ½ | 0.03 | 0.06 | — | — | — | — | 28 | A |
| 26 | | Lower layer | Amount: 1 | 1.0 | — | 1.0 | 3.0 | 0.05 | 0.75 | | |
| Ex. | Ex. 9 | Upper layer | Amount: ½ | 0.03 | 0.06 | — | — | — | — | 28 | A |
| 27 | | Lower layer | Amount: 1 | 1.0 | — | 1.0 | 3.0 | 0.05 | 0.75 | | |
| Com. | Ex. 1 | Upper layer | Amount: ½ | — | — | — | — | — | — | 28 | C |
| Ex. 7 | | Lower layer | Amount: 1 | — | — | — | 3.0 | — | 0.75 | | |
| Com. | Ex. 2 | Upper layer | Amount: ½ | — | — | — | — | — | — | 28 | C |
| Ex. 8 | | Lower layer | Amount: 1 | — | — | — | 3.0 | — | 0.75 | | |
| Com. | Ex. 3 | Upper layer | Amount: ½ | — | — | — | — | — | — | 28 | C |
| Ex. 9 | | Lower layer | Amount: 1 | — | — | — | 3.0 | — | 0.75 | | |
| Com. | Ex. 4 | Upper layer | Amount: ½ | — | — | — | — | — | — | 28 | C |
| Ex. | | Lower layer | Amount: 1 | — | — | — | 3.0 | — | 0.75 | | |
| 10 | | | | | | | | | | | |
| Com. | Ex. 5 | Upper layer | Amount: ½ | — | — | — | — | — | — | 28 | C |
| Ex. | | Lower layer | Amount: 1 | — | — | — | 3.0 | — | 0.75 | | |
| 11 | | | | | | | | | | | |
| Com. | Ex. 6 | Upper layer | Amount: ½ | — | — | — | — | — | — | 28 | C |
| Ex. | | Lower layer | Amount: 1 | — | — | — | 3.0 | — | 0.75 | | |
| 12 | | | | | | | | | | | |
| Com. | Ex. 7 | Upper layer | Amount: ½ | — | — | — | — | — | — | 28 | C |
| Ex. | | Lower layer | Amount: 1 | — | — | — | 3.0 | — | 0.75 | | |
| 13 | | | | | | | | | | | |
| Com. | Ex. 8 | Upper layer | Amount: ½ | — | — | — | — | — | — | 28 | C |
| Ex. | | Lower layer | Amount: 1 | — | — | — | 3.0 | — | 0.75 | | |
| 14 | | | | | | | | | | | |
| Com. | Ex. 9 | Upper layer | Amount: ½ | — | — | — | — | — | — | 28 | C |
| Ex. | | Lower layer | Amount: 1 | — | — | — | 3.0 | — | 0.75 | | |
| 15 | | | | | | | | | | | |

Good elongation of shoots was observed after culture in all Examples 19 to 27. In contrast, in all Comparative Examples 7 to 15 in which culture was carried out in elongation media not supplemented with the plant hormone, silver nitrate, and activated carbon, very little shoot elongation or multiple bud formation was observed and the tissues were exhausted by continuing the culture.

### <Propagation Step>

The shoots elongated and grown by culture in Examples 19 to 27 were each divided with the node, axillary bud, and apical bud portions being left therein, and then transplanted into an elongation medium having the identical composition and cultured. As a result, further new shoots were elongated and multiple buds were generated from the divided shoots, whereby they were propagated. By repeating the propagation step, the shoots could be mass-propagated well.

### Examples 28 to 36, Comparative Examples 16 to 24

### Rooting Step

The shoots induced by culture in Examples 1 to 9 and then grown by subculture were each inserted into a root induction medium (solid medium) and cultured (rooting step). The root induction medium was prepared in such a manner that a plant hormone, silver nitrate, sucrose, and activated carbon at predetermined concentrations shown in the Table 3 below were added to ½ MS medium (disclosed on pages 20 to 36 of Shokubutsu Saibo Kogaku Nyumon (Introduction to Plant Cell Engineering), Japan Scientific Societies Press), the pH of the medium was adjusted to 5.7, and then a gellant was added at 0.75 mass %, followed by autoclave sterilization (at 121° C. for 20 minutes) and then cooling in a clean bench.

The shoots induced by culture in Examples 1 to 9 and then grown by subculture were each inserted into the root induction medium (solid medium) and then cultured for 4 weeks at a culture temperature of 28° C. under illumination at 12.5 μmol/m²/s with a light time of 16 hours. After 4 week culture, the presence or absence of rooting was visually determined. The criteria for determination of the presence or absence are as follows. Moreover, shoot growth rate was calculated using the following equation.

### [Presence or Absence of Rooting]

Present: Rooting was observed in 50% or more of shoots.
Absent: Rooting was observed in less than 50% of shoots.

### [Shoot Growth Rate]

(shoot growth rate (%))={(the number of shoots in which a new bud or growth such as elongation was confirmed)/(the number of shoots transplanted into the root induction medium)}×100

The shoot, the composition of the medium, the culture temperature, the shoot growth rate, and the presence or absence of rooting in Examples 28 to 36 and Comparative Examples 16 to 24 are shown in Table 3.

TABLE 3

| | | | Conc. of | | Conc. of | | | | |
|---|---|---|---|---|---|---|---|---|---|
| | | | silver | Sucrose | activated | Gellant | Culture | | Presence or |
| | | IBA conc. | nitrate | conc. | carbon | conc. | temperature | Shoot growth | absence of |
| | Shoot | (mg/L) | (mg/L) | (mass %) | (mass %) | (mass %) | (° C.) | rate (%) | rooting |
| Ex. 28 | Ex. 1 | 5.0 | 1.0 | 3.0 | 0.05 | 0.75 | 28 | 100 | Present |
| Ex. 29 | Ex. 2 | 5.0 | 1.0 | 3.0 | 0.05 | 0.75 | 28 | 100 | Present |
| Ex. 30 | Ex. 3 | 5.0 | 1.0 | 3.0 | 0.05 | 0.75 | 28 | 100 | Present |
| Ex. 31 | Ex. 4 | 5.0 | 1.0 | 3.0 | 0.05 | 0.75 | 28 | 100 | Present |
| Ex. 32 | Ex. 5 | 5.0 | 1.0 | 3.0 | 0.05 | 0.75 | 28 | 100 | Present |
| Ex. 33 | Ex. 6 | 5.0 | 1.0 | 3.0 | 0.05 | 0.75 | 28 | 100 | Present |
| Ex. 34 | Ex. 7 | 5.0 | 1.0 | 3.0 | 0.05 | 0.75 | 28 | 100 | Present |
| Ex. 35 | Ex. 8 | 5.0 | 1.0 | 3.0 | 0.05 | 0.75 | 28 | 100 | Present |
| Ex. 36 | Ex. 9 | 5.0 | 1.0 | 3.0 | 0.05 | 0.75 | 28 | 100 | Present |
| Com. Ex. 16 | Ex. 1 | — | — | 3.0 | — | 0.75 | 28 | 10 | Absent |
| Com. Ex. 17 | Ex. 2 | — | — | 3.0 | — | 0.75 | 28 | 10 | Absent |
| Com. Ex. 18 | Ex. 3 | — | — | 3.0 | — | 0.75 | 28 | 10 | Absent |
| Com. Ex. 19 | Ex. 4 | — | — | 3.0 | — | 0.75 | 28 | 10 | Absent |
| Com. Ex. 20 | Ex. 5 | — | — | 3.0 | — | 0.75 | 28 | 10 | Absent |
| Com. Ex. 21 | Ex. 6 | — | — | 3.0 | — | 0.75 | 28 | 10 | Absent |
| Com. Ex. 22 | Ex. 7 | — | — | 3.0 | — | 0.75 | 28 | 10 | Absent |
| Com. Ex. 23 | Ex. 8 | — | — | 3.0 | — | 0.75 | 28 | 10 | Absent |
| Com. Ex. 24 | Ex. 9 | — | — | 3.0 | — | 0.75 | 28 | 10 | Absent |

Good rooting was observed after culture in all Examples 28 to 36. The rooted shoots were subcultured by transplanting them into a root induction medium having the identical composition every 4 weeks. By such culture of the rooted shoots, they were regenerated into complete plants. These steps allowed clone seedlings to be mass-propagated well. In contrast, in all Comparative Examples 16 to 24 in which culture was carried out in root induction media not supplemented with the plant hormone, silver nitrate, and activated carbon, rooting of the shoots was not observed and the shoots were exhausted by continuing the culture.

### Examples 37 to 45

### Acclimatization Step

The shoots (young plants) rooted by culture in Examples 28 to 36 were carefully taken out of the culture tubes. The solid culture media attached to the young plants were removed with running water. The young plants were immersed for about 2 to 3 minutes in a disinfectant (0.01 mass % povidone-iodine solution). The young plants were transplanted into pots filled with a cultivation soil. The upper portion of the young plants was covered with a light-permeable plastic bag. The young plants were cultivated under illumination at 12.5 μmol/m²/s with a light time of 16 hours. After the cultivation under the cover for 1 week, the growth of the plants was observed and the cover was gradually removed while checking the state of the plants. When stable growth was observed, the cover was completely removed.

### [Ratings of Survival Rate]

A survival rate was calculated using the following equation and the calculated survival rate was evaluated based on the following rating criteria.

(survival rate (%))={(the number of surviving young plants 2 months after transplanting into the cultivation soil)/(the number of young plants transplanted into the cultivation soil)}×100

### Rating Criteria:

A: Survival rate was 75% or more;
B: Survival rate was at least 25% but not more than 50%;
C: Survival rate was less than 25%.

### Comparative Examples 25 to 33

### Acclimatization Step

An acclimatization step was performed in the same manner as in Examples 37 to 45, except that the young plants were not immersed into the disinfectant and the upper portion of the young plants was not covered with the light-permeable plastic bag.

### Comparative Example 34

### Acclimatization Step

An acclimatization step was performed in the same manner as in Examples 37 to 45, except that the young plants were not immersed into the disinfectant.

The young plant, the concentration of the disinfectant, the presence or absence of the cover, and the rating of survival rate in Examples 37 to 45 and Comparative Examples 25 to 34 are shown in Table 4.

TABLE 4

| | Young | Disinfectant | | Rating of |
|---|---|---|---|---|
| | plant | conc. (mass %) | Cover | survival rate |
| Ex. 37 | Ex. 28 | 0.01 | Present | A |
| Ex. 38 | Ex. 29 | 0.01 | Present | A |
| Ex. 39 | Ex. 30 | 0.01 | Present | A |
| Ex. 40 | Ex. 31 | 0.01 | Present | A |
| Ex. 41 | Ex. 32 | 0.01 | Present | A |
| Ex. 42 | Ex. 33 | 0.01 | Present | A |
| Ex. 43 | Ex. 34 | 0.01 | Present | A |
| Ex. 44 | Ex. 35 | 0.01 | Present | A |
| Ex. 45 | Ex. 36 | 0.01 | Present | A |
| Com. Ex. 25 | Ex. 28 | — | Absent | C |
| Com. Ex. 26 | Ex. 29 | — | Absent | C |
| Com. Ex. 27 | Ex. 30 | — | Absent | C |
| Com. Ex. 28 | Ex. 31 | — | Absent | C |
| Com. Ex. 29 | Ex. 32 | — | Absent | C |
| Com. Ex. 30 | Ex. 33 | — | Absent | C |
| Com. Ex. 31 | Ex. 34 | — | Absent | C |
| Com. Ex. 32 | Ex. 35 | — | Absent | C |
| Com. Ex. 33 | Ex. 36 | — | Absent | C |
| Com. Ex. 34 | Ex. 28 | — | Present | B |

In all Examples 37 to 45, acclimatization occurred well in the acclimatization step and it was demonstrated that young plants were grown well in the natural environment. In contrast, in Comparative Examples 25 to 33 in which no disinfectant was used and the upper portion was not covered, the young plants were grown to a very little extent in the natural environment. Moreover, in a case where the upper portion was only covered without the use of the disinfectant (Comparative Example 34), the young plants were not grown much in the natural environment as compared to Examples 37 to 45.

1. A method of regenerating a rubber tree, comprising
an induction step of culturing a tissue comprising a node, axillary bud or apical bud of a rubber tree in an induction medium containing a plant growth hormone and a carbon source to form a shoot.

2. The method of regenerating a rubber tree according to claim 1, further comprising
an elongation step of culturing the shoot formed through the induction step in an elongation medium containing a plant growth hormone and a carbon source to elongate the shoot.

3. The method of regenerating a rubber tree according to claim 1, further comprising
a rooting step of culturing the shoot formed through the induction step or the shoot elongated through the elongation step in a root induction medium containing a plant growth hormone and a carbon source to root the shoot.

4. The method of regenerating a rubber tree according to claim 3, further comprising
an acclimatization step of treating the shoot rooted through the rooting step with a disinfectant, followed by transplanting the treated shoot into a cultivation soil and acclimatizing the transplanted shoot.

5. The method of regenerating a rubber tree according to claim 1,
wherein the rubber tree is a plant belonging to the genus Hevea.

6. The method of regenerating a rubber tree according to claim 1,
wherein the induction medium contains 0.01 mass % to 0.1 mass % of activated carbon.

7. The method of regenerating a rubber tree according to claim 1,
wherein the induction medium contains 0.1 mg/L to 5.0 mg/L of silver nitrate.

8. The method of regenerating a rubber tree according to claim 3,
wherein the root induction medium contains 0.01 mass % to 0.1 mass % of activated carbon.

9. The method of regenerating a rubber tree according to claim 3,
wherein the root induction medium contains 0.1 mg/L to 5.0 mg/L of silver nitrate.

10. The method of regenerating a rubber tree according to claim 4,
wherein the disinfectant is an iodine-based disinfectant.

11. The method of regenerating a rubber tree according to claim 4,
wherein the acclimatization step comprises, after transplanting the rooted shoot treated with the disinfectant into the cultivation soil, gradually exposing the transplanted shoot to a natural environment to adapt the shoot to the environment for acclimatization.

12. A method of propagating a rubber tree, comprising
an induction step of culturing a tissue comprising a node, axillary bud or apical bud of a rubber tree in an induction medium containing a plant growth hormone and a carbon source to form a shoot.

13. The method of propagating a rubber tree according to claim 12, further comprising
an elongation step of culturing the shoot formed through the induction step in an elongation medium containing a plant growth hormone and a carbon source to elongate the shoot.

14. The method of propagating a rubber tree according to claim 12, further comprising
a propagation step of collecting the shoot formed through the induction step or the shoot elongated through the elongation step, dividing the shoot into multiple shoots, and then culturing the divided shoot in an induction medium containing a plant growth hormone and a carbon source to form a shoot.

15. The method of propagating a rubber tree according to claim 14, further comprising
a rooting step of culturing the shoot formed through the propagation step in a root induction medium containing a plant growth hormone and a carbon source to root the shoot.

16. The method of propagating a rubber tree according to claim 15, further comprising
an acclimatization step of treating the shoot rooted through the rooting step with a disinfectant, followed by transplanting the treated shoot into a cultivation soil and acclimatizing the transplanted shoot.

17. The method of propagating a rubber tree according to claim 12,
wherein the rubber tree is a plant belonging to the genus Hevea.

18. The method of propagating a rubber tree according to claim 12,
wherein the induction medium contains 0.01 mass % to 0.1 mass % of activated carbon.

19. The method of propagating a rubber tree according to claim 12,
wherein the induction medium contains 0.1 mg/L to 5.0 mg/L of silver nitrate.

20. The method of propagating a rubber tree according to claim 15,
wherein the root induction medium contains 0.01 mass % to 0.1 mass % of activated carbon.

21. The method of propagating a rubber tree according to claim 15,
wherein the root induction medium contains 0.1 mg/L to 5.0 mg/L of silver nitrate.

22. The method of propagating a rubber tree according to claim 16,
wherein the disinfectant is an iodine-based disinfectant.

23. The method of propagating a rubber tree according to claim 16,
wherein the acclimatization step comprises, after transplanting the rooted shoot treated with the disinfectant into the cultivation soil, gradually exposing the transplanted shoot to a natural environment to adapt the shoot to the environment for acclimatization.

24. A method of inducing a shoot of a rubber tree, comprising
culturing a tissue comprising a node, axillary bud or apical bud of a rubber tree in an induction medium containing a plant growth hormone and a carbon source to form a shoot.

25. A method of elongating a shoot of a rubber tree, comprising
culturing a shoot of a rubber tree in an elongation medium containing a plant growth hormone and a carbon source to elongate the shoot.

26. A method of rooting a shoot of a rubber tree, comprising
culturing a shoot of a rubber tree in a root induction medium containing a plant growth hormone and a carbon source to root the shoot.

27. A method of acclimatizing a young rubber tree, comprising
treating a young rubber tree with a disinfectant, followed by transplanting the treated young rubber tree into a cultivation soil and acclimatizing the transplanted young rubber tree.
